(19) United States
(12) Patent Application Publication
(10) Pub. No.: US 2020/0316388 A1
(43) Pub. Date: Oct. 8, 2020

(54) Electrical Stimulation Screws

(71) Applicant: Neue Magnetodyn GmbH, Munchen (DE)

(72) Inventors: Markus Rennich, Munchen (DE); Heribert Stephan, Munchen (DE)

(21) Appl. No.: 16/373,933

(22) Filed: Apr. 3, 2019

(51) Int. Cl. A61N 1/372; A61B 17/86; A61N 1/05; A61F 2/28

(52) U.S. Cl. CPC A61N 1/372; A61B 17/8605; A61B 2017/00929; A61N 1/0504; A61F 2/28; A61B 17/8625

(57) ABSTRACT

An electrical stimulation screw, for instance a cortical screw or a locking screw, is configured to generate an electric field in response to a magnetic field. The electrical stimulation screw can include a head body and a head ring that surrounds the head body. The electrical stimulation screw can further include a tip opposite the head along the central anchor axis, and a shaft that connects the head to the tip. In one example, the shaft can define a shaft body that is monolithic with the head body, so as to increase the mechanical strength of the screw without compromising the strength of the electric fields that can be generated by the screw. The head body can define a cavity that is configured to receive a driver so as to rotate the head body and the shaft body about a central anchor axis.

### TECHNICAL FIELD

This disclosure relates generally to bone fixation implants, and in particular relates to electrical stimulation screws that can perform electromagnetic stimulation of a bone fracture to improve healing.

### BACKGROUND

When bones are damaged through trauma, disease, distraction osteogenesis, or orthognathic surgery, bone fixation implants are commonly used to provide anatomical repositioning of bone fragments, to maintain their position, and to ensure union in the desired position. Thus, bone fixation implants are typically designed to achieve proper anatomic fit and function. Additionally, because bone fixation implants often support bones that withstand significant mechanical stress in their anatomic function, implants are often composed of strong and rigid materials. Intramedullary nails are an example of implants that are commonly used to treat fractures in long bones of the body such as fractures in femurs, tibias, and humeri. To treat such fractures, the intramedullary nail is inserted into a medullary canal of the long bone such that the nail spans across one or more fractures to fragments of the long bone that are separated by one or more fractures. Bone anchors are then inserted through the bone and into the intramedullary, thereby fixing the intramedullary nail to the bone. The intramedullary nail can remain in the medullary canal at least until the fracture is fused.

Bone anchors can be configured to electrically stimulate a bone fracture or infection when the bone anchors are exposed to an external electromagnetic field. Such bone anchors can include coils of wire that induce an electrical field across two electrodes or poles. The electrodes are typically separated by an electrical insulator. This can reduce the mechanical torque that can be applied to a bone anchor that is configured as a bone screw. Further, increasing the mechanical torque that can be applied to a given bone screw by increasing its mechanical strength can result in a consequential reduction in the size of the electrical coil and, hence, a reduction in the strength of the electrical fields that can be generated by the bone screw.

### SUMMARY

In an example aspect of the present disclosure, an electrical stimulation screw, for instance a cortical screw or a locking screw, is configured to generate an electric field in response to a magnetic field. The electrical stimulation screw can include a head body and a head ring that surrounds the head body. The electrical stimulation screw can further include a tip opposite the head along the central anchor axis, and a shaft that connects the head to the tip. The shaft can define a shaft body that is monolithic with the head body. The head body can define a cavity that is configured to receive a driver so as to rotate the head body and the shaft body about a central anchor axis. The head ring can define a first electrode or pole that defines a first electrically conductive outer surface of the electrical stimulation screw, and the shaft body can define a second electrode or pole that defines a second electrically conductive outer conductive surface of the electrical stimulation screw that is electrically isolated from the first electrically conductive outer surface. An electrical insulator can be disposed between the head ring and the head body, so as to electrically isolate the first electrode from the second electrode. The electrical insulator can include an epoxy that adheres the head ring to the head body. The electrical insulator can also define the tip of the electrical stimulation screw.

In another example aspect of the present disclosure, an electrical stimulation screw is fabricated that includes a head, a tip, and a shaft that connects the head to the tip. An electrical coil can be wound around a ferromagnetic core to define an electrical coil assembly. The electrical coil assembly can be inserted into a cavity defined by a body of the shaft. A head ring can be placed around a head body of the head, so as to define a first electrode. The head body can be monolithic with the shaft body so as to define a gap between the head ring and the head body. A non-conductive polymer can be injected into the gap between the head ring and the head body, so as to adhere the head ring to the head body. The head ring, head body, shaft body, and electrical coil assembly can be inserted into a form. A non-conducive polymer can be injected into a cavity defined by the shaft body, so as fabricate the tip. A non-conducive polymer can be injected into a cavity defined by the shaft body, such that the polymer surrounds the electrical coil assembly and fills a gap between the electrical coil assembly and the shaft body. A bore can be drilled in the head body. A wire can be placed within the bore so as to electrically connect the head ring with the electrical coil assembly. Further, a non-conducive polymer can be injected into a cavity defined by the shaft body, such that the polymer fills the bore.

The foregoing summarizes only a few aspects of the present disclosure and is not intended to be reflective of the full scope of the present disclosure. Additional features and advantages of the disclosure are set forth in the following description, may be apparent from the description, or may be learned by practicing the invention. Moreover, both the foregoing summary and following detailed description are exemplary and explanatory and are intended to provide further explanation of the disclosure.

### BRIEF DESCRIPTION OF THE DRAWINGS

The foregoing summary, as well as the following detailed description of example embodiments of the present disclosure, will be better understood when read in conjunction with the appended drawings. For the purposes of illustrating the example embodiments of the present disclosure, references to the drawings are made. It should be understood, however, that the application is not limited to the precise arrangements and instrumentalities shown. In the drawings:

FIG. 1 depicts an example embodiment of an electrical stimulation system that includes a pulsed electromagnetic field (PEMF) device and a bone implant system configured to be attached to a bone, wherein FIG. 1 shows a cross section view of the PEMF device and a perspective view of the bone implant system.

FIG. 2 is a cross section view of a portion of the bone shown in FIG. 1 that includes a fractured portion, wherein the bone implant system attached to the bone includes a bone implant and electrical stimulation screws disposed on opposite sides of the fractured portion of the bone.

FIG. 3A is a perspective view of an electrical stimulation screw in accordance with an example embodiment.

FIG. 3B is another perspective view of the electrical stimulation screw shown in FIG. 3A.

FIG. 3C is a cross section of the electrical stimulation screw depicted in FIGS. 3A and 3B.

FIG. 3D is a side elevation view of the electrical stimulation screw depicted in FIG. 3C.

FIG. 3E is an exploded view of the electrical stimulation screw depicted in FIGS. 3A to 3D.

FIG. 4A is a perspective view of another electrical stimulation screw in accordance with another example embodiment.

FIG. 4B is another perspective view of the electrical stimulation screw shown in FIG. 4A.

FIG. 4C is a side elevation view of the electrical stimulation screw depicted in FIG. 4A.

FIG. 4D is a cross section of the electrical stimulation screw depicted in FIG. 4C.

FIG. 4E is an exploded view of the electrical stimulation screw depicted in FIGS. 4A to 4D.

FIG. 5A is a plan view of a ferromagnetic core of the electrical stimulation screws depicted in FIGS. 3A to 4E, in accordance with an example embodiment, wherein the ferromagnetic core defines at least one notch.

FIG. 5B is a plan view of another ferromagnetic core of the electrical stimulation screws depicted in FIGS. 3A to 4E, in accordance with another embodiment, wherein the ferromagnetic core defines at least one flange.

### DETAILED DESCRIPTION

As an initial matter, aspects of the disclosure will now be described in detail with reference to the drawings, wherein like reference numbers refer to like elements throughout, unless specified otherwise. Certain terminology is used in the following description for convenience only, and is not limiting. The term “plurality”, as used herein, means more than one. The terms “a portion” and “at least a portion” of a structure include the entirety of the structure. Certain features of the disclosure that are described herein in the context of separate embodiments may also be provided in combination in a single embodiment. Conversely, various features of the disclosure that are described in the context of a single embodiment may also be provided separately or in any sub-combination.

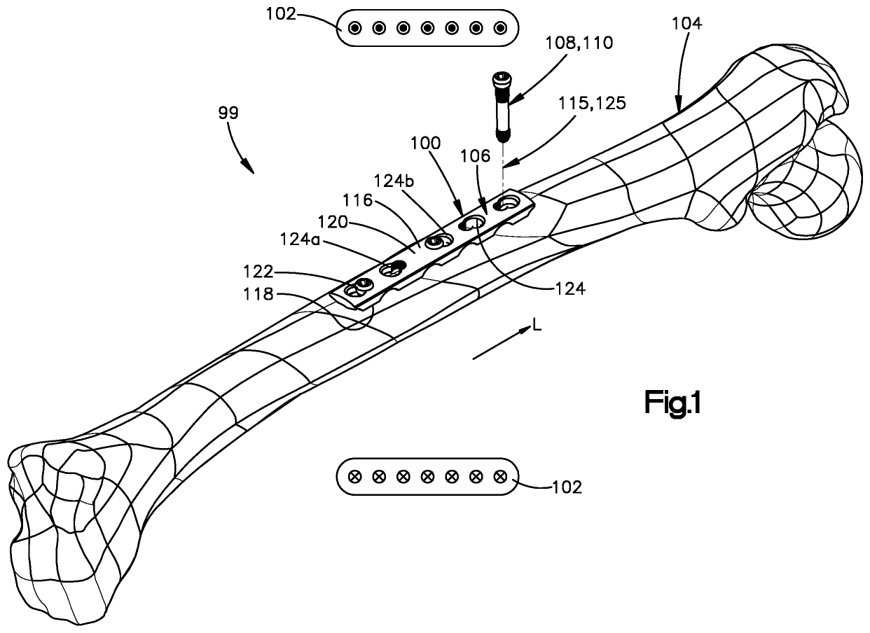

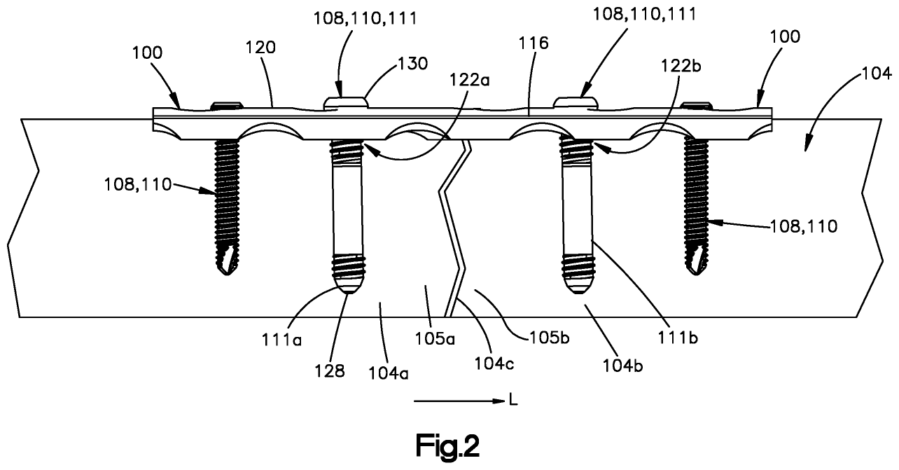

Referring to FIG. 1, an electrical stimulation system 99 includes a bone implant system 100 and a pulsed electromagnetic field (PEMF) device 102. Referring also to FIG. 2, the bone implant system 100 can be configured to be implanted and secured to a bone 104 so as to treat a fractured portion 104c of the bone 104. The bone implant system 100 can be implanted and secured to the bone 104 so as to stabilize a first bone segment 104a of the bone 104 with respect to a second bone segment 104b of the bone 104. The first bone segment 104a and the second bone segment 104b can be separated from each other by the fractured portion 104c of the bone 104. It will be appreciated that the bone 104 can be any bone in the human or animal anatomy suitable for bone implants. Further, while the bone 104 is illustrated having the first bone segment 104a and the second bone segment 104b on opposite sides of the fractured portion 104c, it will be understood that the bone 104 can define any number of fractured portions or bone segments as desired that are configured for fixation using the bone implant system 100.

The bone implant system 100 can include an implant 106, for instance a bone plate or nail, and a plurality of bone anchors 108 that are configured to secure the implant 106 to the underlying bone 104, and in particular to each of the first and second bone segments 104a and 104b. Alternatively, in accordance with another example, the bone implant system 100 includes only the plurality of bone anchors 108, such that the bone anchors 108 are configured to purchase in the bone 104 without the implant 106. The bone anchors 108 can be configured as bone pins or bone screws 110. The bone screws 110 can be configured as electrical stimulation screws 111 configured to respond to a magnetic field so as to generate an electric field.

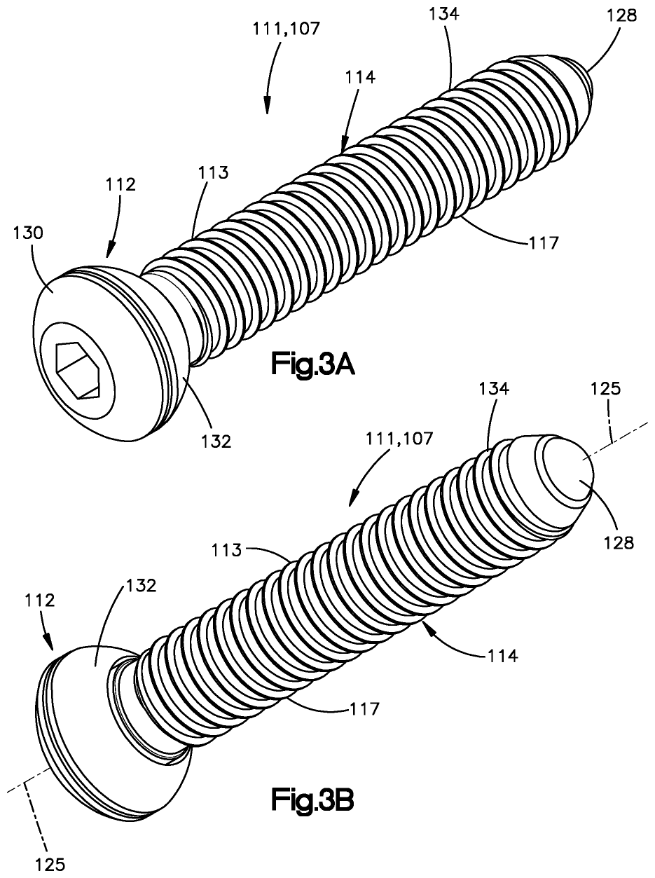

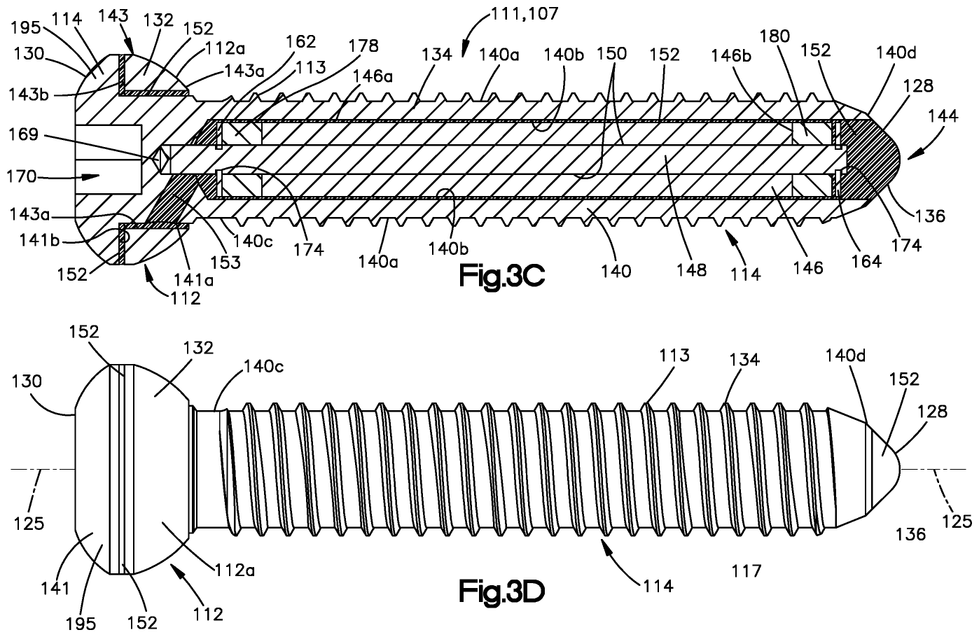

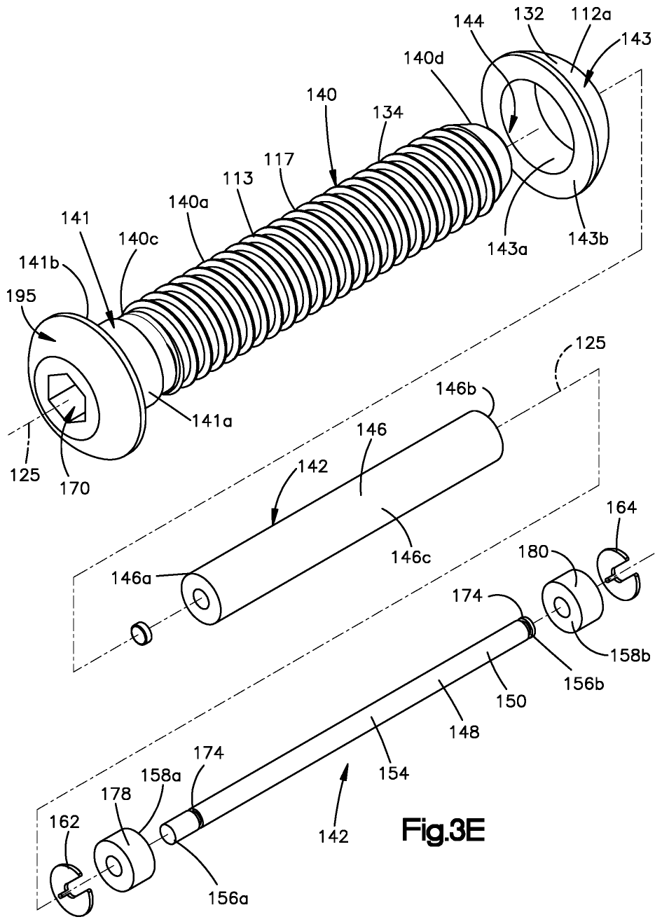

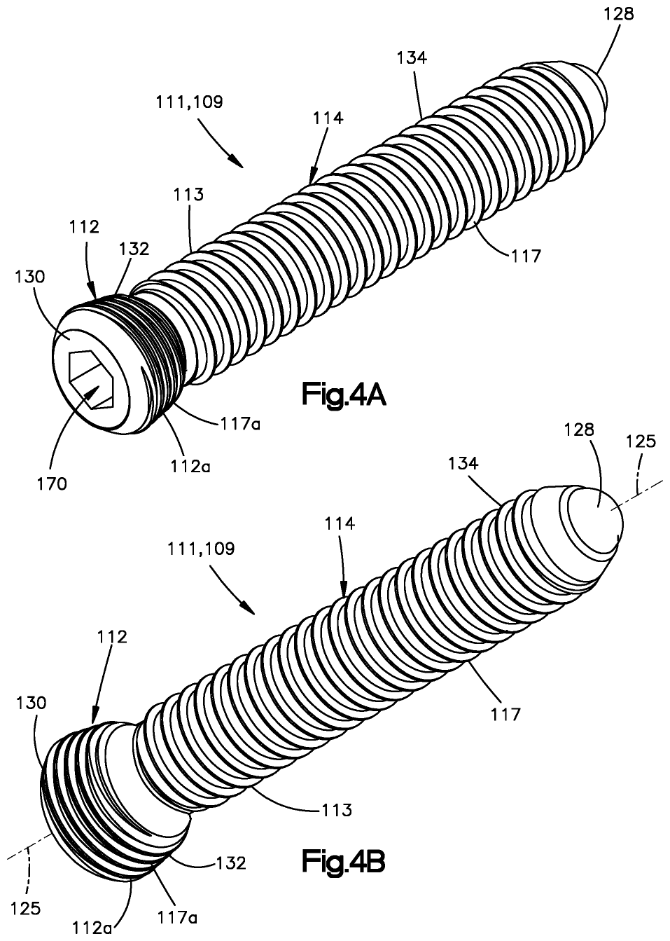

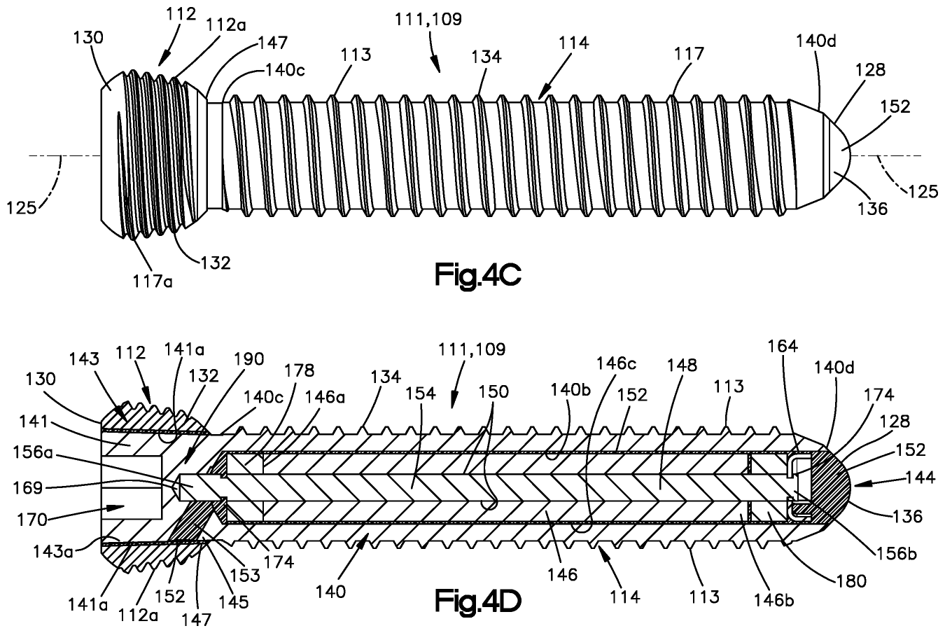

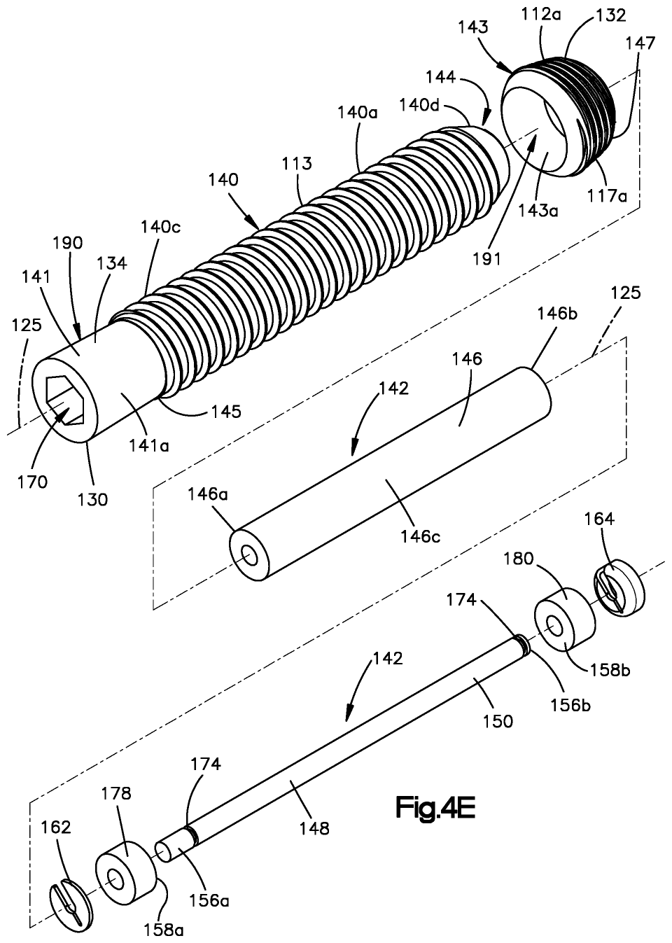

Referring to FIGS. 3A to 4E, the electrical stimulation screws 111 can be configured as an electrical stimulation cortex or cortical screw 107 (e.g., see FIGS. 3A-E), an electrical stimulation locking screw 109 (e.g., see FIGS. 4A-E), or the like. The electrical stimulation screws 111 can include a head 112 and a shaft 114 that extends out with respect to the head 112 along a central anchor axis 125. The shaft 114 can extend directly from the head 112, or can extend from a neck that is disposed between the head 112 and the shaft 114. The shaft 114 can be threaded, such that the electrical stimulation screw 111 includes a threaded shaft 114 extending along the central anchor axis 125, which can also be referred to as a central screw axis. The threaded shaft 114 can be configured to threadedly purchase in the underlying bone 104. For instance, at least a portion, for instance all, of the shaft 114 can be threaded so as to be designed and configured to threadedly mate to cortical bone. Alternatively, or additionally, at least a portion, for instance all, of the shaft 114 can threaded so as to be designed and configured to threadedly mate to cancellous bone. It is appreciated that cancellous bone screws have threads that have a greater pitch than threads of cortical bone screws. Further, the threads of cancellous bone screws typically extend out from the shaft 114 of the bone screw 110 a greater radial distance than the threads of cortical bone screws.

The shaft 114 can define a threaded portion 117 having a thread that is continuous from one end of the shaft 114 to the other end of the shaft 114. Alternatively, a portion of the shaft 114 might not contain threads, such that the shaft 114 does not contain a thread over the entire length of the shaft. The shaft 114 can define threaded portions that are separated by an unthreaded portion, such that the threaded portion 117 is discontinuous from one end of the shaft 114 to the other end of the shaft 114. Alternatively still, in an example, the shaft 114 is not threaded. Referring to FIGS. 4A-E, the head 112 can define a first threaded portion 117a. Thus, the head 112, in particular the first threaded portion 117a, can be configured to lock to the implant 106, such that the electrical stimulation screw 111 defines an electrical stimulation locking screw, such as the electrical stimulation locking screw 109. Alternatively, with reference to FIGS. 3A-E, the head 112 can define an unthreaded portion of the electrical stimulation screw 111.

The bone implant system 100 can include one or more anchors 108 that are configured as electrical stimulation screws 111. The electrical stimulation screws 111 can be configured to secure the implant 106 to the bone 104. The electrical stimulation screws 111 can be configured to respond to a magnetic field so as to generate an electric field. In an example configuration, the bone implant system 100 includes a first electrical stimulation screw 111a adjacent to a first side 105a of the fractured portion 104c, and a second electrical stimulation screw 111b adjacent to a second side 105b of the fractured portion 104c that is opposite the first side 105a of the fractured portion 104c. Thus, the first electrical stimulation screw 111a can be inserted into the first bone segment 104a, and the second electrical stimulation screw 111b can be inserted into the second bone segment 104b, such that the fractured portion 104c is between the first and second electrical stimulation screws 111a and 111b along a longitudinal direction L. The first and second electrical stimulation screws 111a and 111b can be configured to secure the implant 106 to the bone 104, and to respond to a magnetic field so as to generate an electric field between the first and second electrical stimulation screws 111a and 111b. In particular, the first and second electrical stimulation screws 111a and 111b can be configured to respond to a magnetic field so as to generate the electric field at the fractured portion 104c of the bone 104, so as to treat, for instance heal, the fractured portion 104c of the bone 104.

Referring now to FIGS. 1 and 2, the implant 106 can include a body or bone plate 116 that defines an inner plate surface 118 configured to face the underlying bone 104 to which the bone implant system 100 is configured to be attached, and an outer plate surface 120 that is opposite the inner plate surface 118. The implant 106 can further define a plurality of bone fixation holes 122 that extend through the plate 116 from the inner plate surface 118 to the outer plate surface 120. In particular, the plate 116, and thus the implant 106, includes a plurality of internal surfaces 124 that each extend from the outer plate surface 120 to the inner plate surface 118 so as to each define a respective one of the bone fixation holes 122. Each of the bone fixation holes 122 can extend from the outer plate surface 120 to the inner plate surface 118 along a central hole axis 115. The central hole axis 115 can be oriented normal to each of the inner plate surface 118 and the outer plate surface 120. It should be appreciated, of course, that the central hole axis 115 of any of the bone fixation holes 122 can be oriented at an oblique angle with respect to the inner plate surface 118 and outer plate surface 120 as desired.

During a surgical procedure using the bone implant system 100, the shaft 114 of the bone anchor 108, for instance the electrical stimulation screw 111, can be inserted through a respective one of the bone fixation holes 122 and into the underlying bone 104. The electrical stimulation screw 111 can then be rotated, for example about the central anchor axis 125, so as to cause the threaded shaft 114 to be driven into the underlying bone 104 as the threaded shaft 114 threadedly purchases with the underlying bone 104. The threaded shaft 114 can be driven into the underlying bone 104 until the head 112 engages the implant 106. Alternatively, in an example configuration in which the bone implant system 100 does not include the implant 106, such that the electrical stimulation screws 111 are configured as standalone screws, the threaded shaft 114 can be driven into the underlying bone 104 until the head 112 engages the underlying bone 104.

The electrical stimulation screw 111 can be configured as a compression screw whose head 112 is configured to bear against the implant 106 so as to apply a compressive force against the implant 106 toward the underlying bone 104 when the shaft 114 is driven further into the underlying bone 104 after the head 112 has contacted the outer plate surface 120. The shaft 114 can be driven into the underlying bone a sufficient distance until the desired compressive force has been imparted onto the implant 106. The head 112 of the compression screw can be unthreaded. Similarly, at least a portion up to an entirety of the internal surface 124 can be unthreaded.

In another example, the electrical stimulation screw 111 can be configured as the electrical stimulation locking screw 109, which is configured to lock to the implant 106. Therefore, unless otherwise specified, the electrical stimulation screw 111 can refer to the electrical stimulation locking screw 109 or the electrical stimulation cortex screw 107. The electrical stimulation screw 111 can include a head that is externally threaded. The internal surface 124 can be similarly threaded so as to be configured to threadedly mate with the threaded head 112. Accordingly, during operation, the shaft 114 can be inserted through the fixation hole 122 and driven into the underlying bone 104 as described above. In particular, the rotation of the electrical stimulation locking screw 109 causes the threaded head 112 to threadedly mate with the internal surface 124. As a result, the screw head 112 fastens the implant 106 to the underlying bone 104 without applying a compressive force onto the implant 106 against the underlying bone 104. The implant 106 can be spaced from the underlying bone 104 when locked to the head 112. Alternatively, the implant 106 can abut the underlying bone 104 when locked to the head 112. At least a portion of the internal surface 124 can be tapered so as to extend in an axially inward direction, for example toward the central hole axis 115, as the internal surface 124 extends from the outer plate surface 120 toward the inner plate surface 118. Thus, the internal surface 124 can be configured to prevent the head 112 from passing completely through the fixation hole 122. The head 112 can define at least one external thread that is circumferentially continuous about the central anchor axis 125. It should be appreciated, however, that the head 112 can be alternatively constructed in any manner desired so as to threadedly mate with the internal surface 124 as described herein.

According to one embodiment, one or more of the fixation holes 122 of the bone implant 106 can be configured as a variable angle locking hole that is configured to threadedly mate with the electrical stimulation screw 111 at different orientations of the electrical stimulation screw 111 with respect to the central hole axis 115. That is, when the fixation hole 122 is configured as a variable angle locking hole, the plate 116, and thus the implant 106, includes at least one thread that projects out from the internal surface 124 into the fixation hole 122.

The electrical stimulation screw 111 can be configured to be inserted into the fixation hole 122 such that the central anchor axis 125 is at one of a plurality of orientations with respect to the central hole axis 115 within a range of orientations at which the threaded head 112 is configured to threadedly mate with the at least one thread in the fixation hole 122. For instance, the electrical stimulation screw 111 can be configured to be inserted into the fixation hole 122 such that the central anchor axis 125 is at one of a plurality of angles within a range of angles defined by the central anchor axis 125 and the central hole axis 115 at which the threaded head 112 is configured to threadedly mate with the at least one thread in the fixation hole 122. The range of angles can be from approximately zero degrees to approximately thirty degrees. Thus, the range of angles can define a cone of up to approximately sixty degrees. The central anchor axis 125 can be coincident with the central hole axis 115 in one of the orientations in the range of orientations. At least one thread in the fixation hole 122 and the threads of the head 112 can be defined prior to insertion of the electrical stimulation screw 111 into the variable angle locking hole. That is, the internal surface 124 can be designed or configured such that threads are not cut into the bone screw head 112. Similarly, the bone screw head 112 can be designed or configured so as to cut no threads into the internal surface 124.

Referring generally to FIGS. 1 and 2, the bone fixation holes 122 can include first and second bone fixation holes 122a and 122b, respectively, which are spaced from each other along the longitudinal direction L. A first internal surface 124a can extend from the inner plate surface 118 to the outer plate surface 120 so as to define the first bone fixation hole 122a, and a second internal surface 124b can extend from the inner plate surface 118 to the outer plate surface 120 as to define the second bone fixation hole 122b. The first electrical stimulation screw 111a can be sized and configured for insertion into the first bone fixation hole 122a so as to threadedly mate with the first internal surface 124a, and the second electrical stimulation screw 111b can be sized and configured for insertion into the second bone fixation hole 122b so as to threadedly mate with the second internal surface 124b. Thus, the first and second electrical stimulation screws 111a and 111b can be configured to secure the implant 106 to the bone 104 such that the inner plate surface 118 is spaced from the bone 104.

In an example configuration, the first and second electrical stimulation screws 111a and 111b are substantially the same size as each other, and thus the first and second bone fixation holes 122a and 122b can be substantially the same size as each other. The first and second bone fixation holes 122a and 122b can each extend from the inner plate surface 118 to the outer plate surface 120. The first and second bone fixation holes 122a and 122b can configured to be spaced from each other along the longitudinal direction L such that the fractured portion 104c of the bone 104 to which the bone implant system 100 is configured to be attached is disposed between the first and second bone fixation holes along the longitudinal direction L when the implant 106 is positioned over the bone. The first and second bone fixation holes 122a and 122b can be adjacent to each other such that no bone fixation holes 122 are between the first and second bone fixation holes along the longitudinal direction L. It will be understood that the bone fixation holes, and thus the electrical stimulation anchors, can be alternatively located, and the location of bone fixation holes and the electrical stimulation anchors may depend on the size and shape of the fracture being treated.

Referring again to FIGS. 3A-4E, the electrical stimulation screw 111 can define a distal end 128 and a proximal end 130 that is opposite the distal end 128 along the central anchor axis 125. Thus, the proximal and distal ends can refer to portions of the screw 111, and not the position of the screw within the human body, unless otherwise specified. The electrical stimulation screw 111 can be elongate along the central anchor axis 125 between the distal end 128 and the proximal end 130. The head 112 can be disposed at the proximal end 130. Thus, the proximal end 130 can be configured to be disposed adjacent to the outer plate surface 120 of the implant 106 when the implant 106 is secured to the bone 104. The head 112, and thus the proximal end 130, can define a first electrode or pole 132 of the electrical stimulation screw 111. In an example configuration, the first electrode 132 can be configured to contact the implant 106 when the implant 106 is secured to the bone 104. The first electrode 132, and thus the head 112, can define a first electrically conductive outer surface 112a configured to contact the implant 106 when the implant 106 is secured to the bone 104. The distal end 128 can be considered to be an insertion end or leading end. The shaft 114 can define a second electrode or pole 134 of the electrical stimulation screw 111. Further, the distal end 128 can define the second electrode. The second electrode 134, and thus the shaft 114, can include a second electrically conductive outer surface 113 electrically isolated from the first electrically conductive outer surface 112a, such that electrical current induced by a magnetic field is not transferred from the first electrode 132 to the second electrode 134. The shaft 114 can extend between the proximal end 130 and the distal end 128 along the central anchor axis 125 so as to be elongate along the central anchor axis 125.

The electrical stimulation screw 111 can further include a tip 136 that is disposed at the distal end 128. The tip 136 can be opposite the head 112 along the central anchor axis 125. The shaft 114 can connect the head 112 to the tip 136. For instance, the shaft 114 can connect the head 112 to the tip 136, such that there is no electrical short between the first electrode 132 and the shaft 114. The proximal end 130 and the distal end 128 can define opposite outermost ends of the electrical stimulation screw 111. The first and second electrodes 132 and 134 can be composed of electrically conductive material, for instance titanium, stainless steel, or alloys thereof, so as to transfer electrical current. In particular, portions of the head 112 and the shaft can be composed of electrically conductive material, for instance titanium, stainless steel, or alloys thereof, so as to transfer electrical current. The tip 136 can be composed of an injected molded polymer in certain examples.

Referring in particular to FIGS. 3C, 3E, 4D, and 4E, the shaft 114 can include a shaft body 140 and an electrical coil assembly 142 disposed within the shaft body 140. The shaft body 140 can include an outer surface 140a and an inner surface 140b opposite the outer surface 140a. For example, the shaft body 140 can define a cavity 144 within which a portion or all of the electrical coil assembly 142 can be disposed. The second electrically conductive outer surface 113 defined by the second electrode 134 can include at least a portion, for instance all, of the outer surface 140a of the shaft body 140. The shaft body 140 can be composed of titanium, stainless steel, or alloys thereof. The head 112 define include a head body 141 that is monolithic with the shaft body 140. Thus, the head body 141 can be disposed of titanium, stainless steel, or alloys thereof.

With continuing reference to FIGS. 3C, 3E, 4D, and 4E, the head 112, in particular the head body 141, can define a cavity 170 that can be sized so as to receive a driver, for instance a screw driver of a surgical drill. Thus, the head body can define the cavity 170 configured to receive a driver so as to rotate the head body 141 and the shaft body 140 about the central anchor axis 125. The driver can be inserted into the cavity 170 and sized so as to rotate the electrical stimulation screw 111 about the central anchor axis 125. In particular, the driver can rotate the head body 141, and thus the shaft body 140, about the central anchor axis 125. Torque applied to the head body 141 can be transferred to the shaft body 140, for example, because the head body 141 can be monolithic with the shaft body 140. The shaft body 140 can also define a shaft body proximal end 140c and a shaft body distal end 140d opposite the shaft body proximal end 140c along the central anchor axis 125. The shaft body proximal end 140c can abut the head 112, in particular the head body 141. The shaft body distal end 140d can be attached to the tip 136.

The electrical stimulation screw 111 can be elongate from the proximal end 130 to the distal end 128. For instance, the screw can be substantially elongate along the central anchor axis 125 that extends from the proximal end 130 to the distal end 128. It will be appreciated that the central anchor axis 125 of the electrical stimulation screw 111 can be straight or curved. Thus, the shaft 114 can be straight or curved as it extends along the central anchor axis 125 from the head 112 to the tip 136.

Referring in particular to FIGS. 3C, 3E, 4D, and 4E, the electrical stimulation screw 111, in particular the head 112, can include a head ring 143 that defines the first electrically conductive outer surface 112a. The head ring 143 can wrap around the head body 141. Thus, the head ring 143 can surround at least a portion of the head body 141. The head ring 143 can define the first electrode 132 that defines the first electrically conductive outer surface 112a of the electrical stimulation screw 111, and the shaft body 140 can define the second electrode 134 that defines the second electrically conductive outer surface 113 of the electrical stimulation screw 111 that is electrically isolated from the first electrically conductive outer surface 112a.

The head ring 143 can be spaced from the head body 141 along a direction that is radially outward from the central anchor axis 125. In particular, the head ring 143 can define a first or inner head ring surface 143a that faces the central anchor axis 125. The head ring 143 can further define the first electrically conductive outer surface 112a that is opposite the inner head ring surface 143a. The head body 141 can define a first head body surface 141a that faces away from the central anchor axis 125. Thus, the inner head ring surface 143a and the first head body surface 141a can face each other. Further, the inner head ring surface 143a and the first head body surface 141a can be spaced from each other along the direction that is radially outward from the central anchor axis 125. The electrical stimulation screw 111 can further include an electrical insulator 152, for instance an insulative epoxy or other non-conductive polymer, which adheres the first head body surface 141a to the inner head ring surface 143a. Thus, the electrical insulator can electrically isolate the first head body surface 141a from the inner head ring surface 143a. The electrical insulator 152 between the head ring 143 and the head body 141 can include an epoxy resin or other synthetic material so as to bio-compatibly shield the head ring 143 from the head body 141.

Referring in particular to FIGS. 3C and 3E, the head body 141 of the electrical stimulation screw 111, for instance the head body 141 of electrical stimulation cortex screw 107, can further define a second head body surface 141b that faces the tip 136. Further, the head ring 143 can further define a second or top head ring surface 143b that faces the second head body surface 141b. In particular, the second head body surface 141b and the top head ring surface 143b can be spaced from each other along the central anchor axis. Thus, the top head ring surface 143b can define a plane that is substantially perpendicular to a plane defined by the inner head ring surface 143a. The electrical insulator 152, for instance an insulative epoxy or non-conductive polymer, can adhere the second head body surface 141b to the top head ring surface 143b. Thus, the electrical insulator can electrically isolate the second head body surface 141b from the top head ring surface 143b. The electrical insulator 152 can be disposed between the head body 141 and the head ring 143 along the central anchor axis 125, so as to electrically isolate the head body 141 and the head ring 143 from each other. Thus, the electrical insulator 152 can electrically isolate the head ring 143, in particular the first electrically conductive outer surface 112a, from the second electrically conductive outer surface 113, thereby electrically isolating the first electrode 132 from the second electrode 134. Further, the electrical insulator 152 can define the tip 136.

With continuing reference to FIGS. 3C and 3E, the head body 141 of the electrical stimulation cortex screw 107 can define a stop cap 195 for the head ring 143. In particular, the stop cap 195 can include the second head body surface 141b that can stop the head ring 143 from moving to the proximal end 130. In an example assembly process, the head ring 143 can be pushed into place onto the head body 141 toward the proximal end 130 from the distal end 128, until the top head ring surface 143b contacts the second head body surface 141b, such that the head ring 143 is stopped by the second head body surface 141b. Continuing with the example assembly process, when the head ring 143 is pushed toward the proximal end 130 so that the head ring 143 is stopped from moving further along the central anchor axis 125 by the head body 141, the electrical insulator 152 can be injected between the head body 141 and the head ring 143, in particular between the second head body surface 141b and the top head ring surface 143b, so as to hold the head ring 143 in place with respect to the head body 141. Thus, the head ring 143 of the electrical stimulation cortex screw 107 can be form closed to the head body 141 by the electrical insulator 152.

Without being bound by theory, it is recognized herein this arrangement of the head ring 143 and the head body 141 including the stop cap 195 can absorb axial forces in axial directions (or the directions along the central anchor axis 125). For example, when an axial force toward the proximal end 130 from the distal end 128 acts on the head ring 143, the electrical insulation 152 between the second head body surface 141b and the top head ring surface 143b can absorb the axial force because it is located between the planar surfaces 141b and 143b, thereby reducing or eliminating tensile forces on the electrical insulator 152. Thus, the stop cap 195 can be configured to absorb force applied to the head ring 143 in an axial direction from the distal end 128 toward the proximal end 130.

Referring now in particular to FIGS. 3E, 4E, 5A, 4D and 5B, the electrical coil assembly 142 can include a ferromagnetic core 148 and an electrical coil 146 arranged, for instance wound, about or around the ferromagnetic core 148. The coil 146 can include an electrically conductive wire that can be wound around the ferromagnetic core 148. In an example, the electrically conductive wire can be wound about the central anchor axis 125. The ferromagnetic core 148 can define an external surface 150, such that the electrically conductive wire, and thus the coil 146, abuts the external surface 150. The electrical coil 146 and the ferromagnetic core 148 can be disposed within the electrical insulator 152. The electrical coil 146 can be wound around the ferromagnetic core 148 so as to contact the electrical insulator 152. Thus, the electrical insulator 152 can be disposed between the electrical coil assembly 142, in particular the electrical coil 146 and the shaft body 140 along the direction that is radially outward from the central anchor axis. In particular, the electrical insulator 152 can contact the inner surface 140b of the shaft body 140 and an outer surface 146c of the electrical coil. In an example configuration, an electrically insulative epoxy is injected into the cavity 144 of the shaft body 140 so as to form the electrical insulator 152, and thus the tip 136.

Referring in particular to FIGS. 3C and 4D, 5A, 5B the head body 141 can define a bore or channel 153 from the first head body surface 141a to the central anchor axis 125. The electrical stimulation screw 111 can include a wire that extends through the bore 153 so as to electrically connect the head ring 143 to a first or coil proximal end 146a (or starting end) of the electrical coil 146. Referring also to FIG. 3E, the electrical coil 146 can define the coil proximal end 146a that can be proximate to the head 112, and a second or coil distal end 146b (or terminating end) opposite the coil proximal end 146a along the central anchor axis 125. The coil distal end 146b can be proximate to the tip 136. The electrical insulator 152 can also be injected or otherwise disposed within the bore 153 so as to electrically isolate the wire from the head body 141.

Referring to FIGS. 3E, 4E, 5A and 5B, the shaft 114 can include the electrical coil assembly 142, and thus the core 148. The core 148 can define a core body 154 and a core proximal end 156a disposed at a first end of the core body 154. The core proximal end 156a can be attached to the head body 141, and thus to the head 112, so as to be configured to transfer torque applied to the head 112 about the central anchor axis 125, to the electrical coil assembly 142, so that the electrical coil assembly 142 rotates with the head body 141 and the shaft body 140. The core 148 can further include a core distal end 156b opposite the core proximal end 156a. The core 148, for instance the core proximal end 156a, can be attached to the head 112 of the electrical stimulation screw 111, for instance by a press-fit, so as to mechanically connect the core 148, and thus the electrical coil assembly 142, with the head 112, in particular the head body 141. The core proximal end 156a can be disposed within the electrical insulator 152. Additionally, or alternatively, the head 112 can include the electrical insulator 152 that can include an epoxy that adheres the core 148, in particular the core proximal end 156a, to the head body 141. Further, the shaft 114 can include the electrical insulator 152 that can include an epoxy between the electrical coil 146 and the shaft body 140, in particular the inner surface 140b of the shaft body 140, so as to adhere the electrical coil assembly 142 to the shaft body 140. Thus, the torque applied to the head 112 can be transferred to the shaft 114 so that the electrical coil assembly 142, the head body 141, and the shaft body rotate as one about the central anchor axis 125.

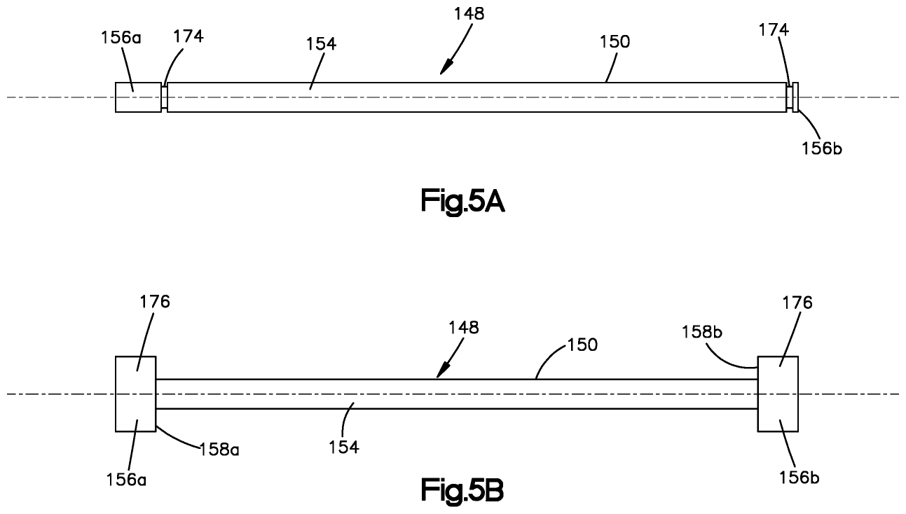

The head 112, in particular the head body 141, can define a slot 169 sized to receive the proximal end 156a of the core 148. Referring to FIG. 5B, the core proximal end 156a can extend outward from the central anchor axis 125 with respect to the core body 154 so as to define a first flange 176. The first flange 176 can insert into the slot 169 so as to lock the core 148 into place with respect to the head body 141. It will be understood that the core proximal end 156a can be sized as desired so as to lock into place with respect to the head body 141. In some example, the core distal end 156b can extend outward from the central anchor axis 125 with respect to the core body 154 so as define as a second flange 176. The second flange 176, and thus the core distal end 156b, can be sized as desired so as to attach to the tip 136.

The core distal end 156b can be disposed at a second end of the core body 154 that is opposite the first end of the core body 154 along the central anchor axis 125. The core 148, in particular the core distal end 156b of the core 148, can be attached to the tip 136. The core 148, for instance the core distal end 156b, can be attached to the tip 136 of the electrical stimulation screw 111, for instance by press-fit, so as to mechanically connect the electrical coil assembly 142 with the tip 136.

Referring in particular to FIG. 5A, the core 148 can include at least one notch or groove 174, for instance a first notch 174 and a second notch 174. In an example, the first notch 174 can be proximate to the core proximal end 156a, and the second notch 174 can be proximate to the core distal end 156b. The core body 154 can be elongate along the central anchor axis, and can define a substantially cylindrical shape, though it will be understood that the core body 154 can be alternatively shaped as desired. In an example, the core body 154, in particular the external surface 150 of the core 148, can define a diameter proximate to the core proximal end 156a that is less than the diameter defined by other portions, for instance portions adjacent to the first notch 174, of the core 148, so as to define the first notch 174. Similarly, the core body 154, in particular the external surface 150 of the core 148, can define a diameter proximate to the core distal end 156b that is less than the diameter defined by other portions, for instance portions adjacent to the second notch 174, of the core 148, so as to define the second notch 174. In an example, the first notch 174 can define a diameter that is equal to the diameter of the second notch 174, though it will be understood that the diameters of the first and second notches 174 can vary as compared to each other as desired. Further, the core 148 can include zero notches 174, or any number of notches 174 as desired. In an example, the core 148 includes the first notch 174 and the second notch 174, and the electrical coil 146 is disposed between the first notch 174 and the second notch 174 along the central anchor axis.

The distance between the coil proximal end 146a and the coil distal end 146b along the central anchor axis 125 can define a length of the coil 146 along the central anchor axis 125, which can be shorter than a length of the core 148 along the central anchor axis 125 that can be defined by the distance between the core proximal end 156a and the core distal end 156b along the central anchor axis 125. The electrical coil assembly 142 can further include one or more spacers 178, for instance a first spacer 178 and a second spacer 180 spaced from the first spacer 178 along the central anchor axis 125. In an example, the first spacer 178 can be proximate to the core proximal end 156a, and the second spacer 180 can be proximate to the core distal end 156b. The first spacer 178 can be between, for instance adjacent to, the first notch 174 and the coil proximal end 146a along the central anchor axis. Similarly, the second spacer 180 can be between, for instance adjacent to, the coil distal end 146b and the second notch 174 along the central anchor axis.

The first and second spacers 178 and 180 can be made of an elastic material, for instance a foam or silicone, so as to absorb shrinkage of epoxy resin (e.g., insulator 152) during curing, following an injection molding. Thus, the length of the electrical coil 146 from the coil proximal end 146a to the coil distal end 146b along the central anchor axis 125 can be varied, for example, so as to optimize magnetic amplification by the core 148. It is recognized herein that magnetic amplification can be lower at the ends of the core 148, for instance the core proximal end 156a and the core distal end 156b, as compared to a center of the core 148 between the core proximal end 156a and the core distal end 156b along the central anchor axis. Thus, the first and second spacers 178 and 180 can allow the electrical coil 146 to define a length along the central anchor axis 125 that is less than a length defined by the core 148 along the central anchor axis 125. It is further recognized herein that, in some cases, mechanical stresses on the core 148 can have a negative effect on magnetostriction, on the saturation of the magnetic flux density, and on harmonics. Without being bound by theory, the first and second spacers 178 and 180 can reduce the mechanical tension on the core body 154, so as to achieve a higher material specific magnetic field, thereby increasing the electric field generated by the electrical stimulation screw 111. It is recognized herein that reducing or preventing torque forces on the core 148 can enable the core 148 to maintain its magnetic properties, because, in some cases, the core 148 can break down due to torque.

The electrical coil assembly 142 can further include one or more locking caps, for instance a first locking cap 162 and a second locking cap 164 spaced from the first locking cap along the central anchor axis 125. The first cap 162 can be supported by the first notch 174, and the second cap 164 can be supported by the second notch 174. Thus, the first cap 162 can be proximate to the core proximal end 156a, and the second cap 164 can be proximate to the core distal end 156b. The second cap 164 can be disposed between the coil distal end 146b of the electrical coil 146 and the tip 136 along the central anchor axis 125. In particular, the first and second caps 162 and 164 can fasten or otherwise attach to the respective notches 174. The first spacer 178 can be between, for instance adjacent to, the first cap 162 and the coil proximal end 146a along the central anchor axis 125. The second spacer 180 can be disposed between, for instance adjacent to, the coil distal end 146b and the second cap 164 along the central anchor axis 125. Thus, the first and second caps 162 and 164 can operate so as to limit expansion of the first and second spacers 178 and 180. The first and second caps 162 and 164 can be made of a soft magnetic material, so as to amplify the magnetic flux at the ends 156a and 156b of the core 148. Therefore, the first and second caps 162 and 164 can extend the length of the core 148. In some cases, though, the first and second caps 162 and 164 do not have the same magnetic saturation as the core body 154, and therefore may be fabricated from a different (or the same) material as the core body 154. It will be understood that the electrical coil assembly 142 can include one or both of the first and second locking caps 162 and 164 as desired. For example, the same locking cap, for instance the first locking cap 162 or the second locking cap 164, can be disposed at both ends of the core 148.

Referring in particular to FIGS. 4D and 4E, an end of the wire that comprises the electrical coil 146 can be electrically connected to the core 148. As described above, the electrical stimulation screw 111 can include a wire that extends through the bore 153 so as to electrically connect the head ring 143 to the proximal end 146a (or starting end) of the electrical coil 146. By way of example, the distal end 146b of the coil 146 can be electrically connected, for instance directly connected, to the external surface 150 of the core 148. Alternatively, or additionally, the distal end 146b of the coil 146 can be connected to the first or second locking cap 162 or 164 that can be electrically and mechanically connected to the core 148. Further, the core proximal end 156a can be received by the slot 169, such that the core proximal end 156a, and thus the core 148, is electrically and mechanically connected to the head body 141. Further still, the head body 141 can be monolithic with, and thus electrically connected to, the shaft body 140. As result, the head body 141 and the shaft body 140 can define the second electrode 134.

Referring again to FIG. 5B, in an example, the core 148 can include the first and second flanges 176 instead of the first and second locking caps 162 and 164. The core proximal end 156a, in particular the first flange 176, can further include a proximal surface 158a that faces the distal end 128 of the electrical stimulation screw 111. The core distal end 156b, in particular the second flange 176, can further include a distal surface 158b that faces the proximal end 130 of the electrical stimulation anchor. Referring to FIGS. 3C and 4D, in some cases, the first spacer 178 can include the proximal surface 158a. Further, the second spacer 180 can define the distal surface 158b, though it will be understood that the electrical stimulation screw can include any number of spacers, for instance zero or one, as desired. The proximal surface 158a and the distal surface 158b can face opposite directions as each other along the central anchor axis 125. For instance, the distal surface 158b can face the proximal surface 158a. In some examples, the proximal surface 158a, distal surface 158b, and the core body 154 can support the coil 146. Thus, in some cases, the first spacer 178, the second spacer 180, and the core body 154 can support the coil 146. The coil 146 can be wound from the proximal surface 158a, to the distal surface 158b, on the external surface 150 and about the central anchor axis 125. The coil 146 can be wound in a clockwise or counterclockwise direction so as to define opposite poles at the coil proximal end 146a and the coil distal end 146b. The external surface 150 of the core body 154 can extend from the proximal surface 158a to the distal surface 158b.

Referring in particular to FIGS. 4D and 4E, the second cap 164 can be electrically conductive and can be in contact with the shaft body 140, in particular the inner surface 140b of the shaft body 140. The second cap 164 can also be in contact with the core 148, in particular the core distal end 156b, and the shaft body 140, so as to electrically connect the coil 146 with the shaft body 140, and thus the coil distal end 146b with the second electrode 134. The head body 141 of the electrical stimulation locking screw 109 can define a first or body truncated cone 190. For example, the head body 141 can include the first head body surface 141a that faces away from the central anchor axis 125, and the head body surface 141a can converge toward the central anchor axis 125. For example, the head body surface 141 can converge toward the central anchor axis 125 as the head body surface 141 approaches the distal end 128 from the proximal end 130. For example, the body truncated cone 190, and thus the head body 141, can define a base diameter at the proximal end 130 of the screw 109, and a frustum diameter that is less than the base diameter at a head body distal end 145 of the head body 141 that is opposite the proximal end 130 of the screw 109 along the central anchor axis 125. The head body distal end 145 can abut the shaft body proximal end 140c. Thus, the body truncated cone 190 can define its frustum diameter proximate to the shaft 114, for instance proximate to the shaft body proximal end 140c. It will be understood that the head body 141 can be alternatively shaped as desired, such that the body truncated cone 190 can define distances other than the base diameter and the frustum diameter at the proximal end 130 and the head body distal end 145, respectively.

With continuing reference to FIGS. 4D and 4E, the head ring 143 of the electrical stimulation locking screw 109 can define a second or ring truncated cone 191 that is sized so as to surround, for instance receive, the body truncated cone 190 of the head body 141. The head ring 143 can include the inner head ring surface 143a that faces the central anchor axis 125, and the inner head ring surface 143a can converge toward the central anchor axis 125. For example, the inner head ring surface 143a can converge toward the central anchor axis 125 as the inner head ring surface 143 approaches the distal end 128 from the proximal end 130. The ring truncated cone 191, and thus the head ring 143, can define a base diameter at the proximal end 130 of the screw 109, and a frustum diameter that is less than the base diameter at a head ring distal end 147 of the head ring 143 that is opposite the proximal end 130 of the screw 109 along the central anchor axis 125. The head ring distal end 147 can be adjacent to the shaft body proximal end 140c. Thus, the ring truncated cone 191 can define its frustum diameter proximate to the shaft 114, in particular proximate to the shaft body proximal end 140c. It will be understood that the head ring 143 can be alternatively shaped as desired, such that the ring truncated cone 191 can define distances other than the base diameter and the frustum diameter at the proximal end 130 and the head ring distal end 147, respectively.

The first head body surface 141a can converge toward the central anchor axis 125 so as to define an angle with respect to the central anchor axis 125 that can be substantially equal to an angle defined by the inner head ring surface 143a with respect to the central anchor axis 125. Further, the base diameter defined by the ring truncated cone 191 of the head ring 143 can be greater than the base diameter defined by the body truncated cone 190 of the head body 141, and the frustum diameter defined by the ring truncated cone 191 of the head ring 143 can be greater than the frustum diameter defined by the body truncated cone 190 of the head body 141. Further, the frustum diameter defined by the head ring 143 can be greater than the base diameter defined by the head body 141. Thus, in an example assembly or mounting process, the head ring 143 can be pushed into place onto the head body 141 from the proximal end 130 toward the distal end 128, until the head body distal end 145 is aligned with the head ring distal end 147 along a direction that extends radially outward from the central anchor axis 125. The head ring distal end 147 can move along the central anchor axis from the proximal end 130 to the head body distal end 145. When the head ring 143 is mounted to the head body 141 in this manner, it will be appreciated that the shaft 114 can be sized as desired, as the shaft 114 does not receive the head ring 143. For example, the shaft 114 can define a diameter that is less than, equal to, or greater than the base diameter of the head ring 143. Thus, in an alternative example in which the diameter of the shaft 114 is less than the frustum diameter of the head ring 143, the head ring 143 can be pushed into place onto the head body 141 from the distal end 128 to the proximal end 128 along the central anchor axis 125.

Continuing with the example mounting process, when the head ring 143 is pushed toward the distal end 128 so that the head body distal end 145 is aligned with the head ring distal end 147, the head ring 143 and the shaft body 140 can be held into place with respect to each other using a casting mold. While the head ring 143, head body 141, and shaft body 140 are in the casting mold, the electrical insulator 152 can be injected between the head body 141 and the head ring 143, in particular between the first head body surface 141a and the inner head ring surface 143a, so as to hold the head ring 143 in place with respect to the head body 141. Thus, the head ring 143 can be form closed to the head body 141 by the electrical insulator 152.

Without being bound by theory, it is recognized herein that this conical arrangement of the head body 141 and the head ring 143 can absorb axial forces in an axial direction (or the direction along the central anchor axis 125). For example, when a force toward the proximal end 130 from the distal end 128 acts on the head ring 143, the cone 191 of the head ring 143 can absorb the force by pushing against the electrical insulation 152, which in turn can push against the head body 141. Thus, the head ring 143 can be resistant to being moved or pushed off the head body 141 along the central anchor axis 125. In particular, the body truncated cone 190 can be configured to absorb force applied to the head ring 143 in an axial direction from the distal end 128 to the proximal end. It is recognized herein that the arrangement of the ring truncated cone 191 surrounding the body truncated cone 190 can resist movement of the head ring 143 with respect to the head body 141, in some cases, better than an arrangement in which the head ring defines a cylinder that wraps around a cylinder defined by the head body.

In an example manufacturing process for fabricating an electrical stimulation screw, such as the electrical stimulation locking screw 109 or the electrical stimulation cortex screw 107, the electrical coil 146 can be wound around the ferromagnetic core 148 to define the electrical coil assembly 142. The electrical coil assembly 142 can be inserted into the cavity 144 defined by the shaft body 140 of the shaft 114. The head ring 143 can be positioned around the head body 141 that is monolithic with the shaft body 140 so as to define a gap between the head ring 143 and the head body 141. For example, the head ring 143 can be moved toward the distal end 128 from the proximal end 130 along the central anchor axis 125, or the head ring 143 can be moved to the proximal end 130 from the distal end 128. A non-conductive polymer can be injected into the gap between the head ring 143 and the head body 141, so as to adhere the head ring 143 to the head body 141. It will be understood that the gap can be filled with epoxy resin or other synthetic material so as to bio-compatibly shield the head ring 143 from the head body 141. In some cases, the head ring 143, the head body 141, the shaft body 140, and the electrical coil assembly 142 can be set in a form or casting mold. While in the form, the non-conductive polymer can be injected into the cavity 144 defined by the shaft body 140, so as to fabricate the tip 136. Additionally, or alternatively, a non-conductive polymer can be injected into the cavity 144 defined by the shaft body 144 such that the polymer surrounds the electrical coil assembly 142 and fills a gap between the electrical coil assembly 142 and the shaft body 140, so as to define the electrical insulator 152. In an example, the bore 153 can be drilled in the head body 141. An electrically conductive wire can be placed within the bore 153 so as to electrically connect the head ring 143 with the electrical coil assembly 142. Further, a non-conductive polymer can be injected into the cavity 144 defined by the shaft body 140 such that the polymer fills the bore 153, thereby electrically isolating the wire from the head body 141.

Thus, the core proximal end 156a can be attached to the head 112, and the core distal end 156b can be attached to the tip 136 that is opposite the head 112 along the central anchor axis 125. In an example, epoxy is injected at the tip 136 to mold the electrical insulator 152 around the coil assembly 142. Before injection molding the electrical insulator 152 in a form, in an example, the electrically conductive portions of the electrical stimulation screw 111 are positioned in a form or mold. The first and second caps 162 and 164, with or without the first and second spacers 178 and 180, can hold the coil 146 in place relative to the core 148 in the form, so that the electrical coil assembly 142 is centered within the electrical insulator 152 after injection molding the insulator material to form the electrical insulator 152 around the electrical coil assembly 142.

In operation, referring also to FIG. 2, the bone implant system 100 can be exposed to a magnetic field that is generated by the PEMF device 102, so as to generate an electric field between the first and second electrical stimulation screws 111a and 111b. In another example, the electrical stimulation screw 111 can be exposed to a magnetic field that is generated by the PEMF device 102, so as to generate an electrical field between the first electrode 132 and the second electrode 134. The magnetic field generated by the PEMF device 102 can be a dynamic field that induces an electric current in the electrical coil 146. In an example implementation, a 0.5 to 5 mT, for instance 3 to 5 mT, magnetic field can be generated as a continuous sinusoidal signal from 20 to 100 Hz, for instance 15 to 30 Hz. The magnetic field can induce a voltage in the screws from 50 to 700 mV_{RMS}. In an example, the peak maximum value can be about 1 V, which is below the disassociate voltage of water (1.2V) and other bodily fluids, such that no toxic substances are produced. In particular, the PEMF device 102 can include one or more coils that can function as a primary coil, and the coils 146 can function as secondary coils when exposed to the magnetic field generated by the primary coil of the PEMF device 102. In an example configuration, the electric coil 146 of the first electrical stimulation screw 111a can be wound in a direction that is opposite the direction in which the coil 146 of the second electrical stimulation screw 111b is wound. Thus, the second electrode 134 of the first electrical stimulation screw 111a can have a polarity that is opposite the polarity of the second electrical stimulation screw 111b. Thus, for example, the first and second electrical stimulation screws 111a and 111b can be configured to respond to a magnetic field so as to generate an electric field from the distal end 128 of one of the first and second electrical stimulation screws 111a and 111b to the distal end 128 of the other of the first and second electrical stimulation screws 111a and 111b.

In some cases, the plate 116 can electrically connect the first electrode 132 of the first electrical stimulation locking screw 111a with the first electrode 132 of the second electrical stimulation locking screw 111b. It is recognized herein that this configuration can increase the predictability and reliability of the electric field that is generated by the first and second electrical stimulation screws 111a and 111b. In some cases, having the first and second electrodes bonded together proximate to the head 112 can reduce the mechanical strength of the screw, thereby reducing the torque that can be applied to the screw. Without being bound by theory, it is also recognized herein that the above-described electrical stimulation screws can have a torque applied thereto that is only limited by the mechanical properties of the head body 141 and the shaft body 140 (e.g., titanium or stainless steel), rather than being limited by the mechanical properties of a bonding agent, because the head body 141 and the shaft body 140 can define the drive and can be monolithic with each other. Thus, as described above, the head body 141 can be glued to the head ring 143 using an insulating epoxy so as to define a glue joint, but this glue joint is not under stress when the screw is driven so as to rotate about the central anchor axis 125. Further, the head ring 143 and head body 141 can be configured to absorb axial forces as described herein, such that the glue joint (e.g., the electrical insulator 152) can avoid tensile forces. Further still, it is recognized herein that the above-described electrical stimulation screws can define a volume within the shaft body 140 that is greater than existing electrical stimulation screws, thereby enabling a larger electrical coil assembly 142 to be disposed within the shaft body 140, so as to strengthen the electric field that is generated as compared to other electrical stimulation screws having a small volume within their respective shaft bodies. Thus, electrical stimulation screws can be manufactured with strengthened electric field capabilities, without increasing the external physical properties of the screw. Similarly, smaller screws can be manufactured without sacrificing the size of the electrical coil, and thus the strength of the electric field. That is, smaller screws can produce stronger electric fields.

In response to the magnetic field generated by the PEMF device 102, by way of example, an electrical current can be induced in the coil 146 between the proximal end 130 and the distal end 128. For example, the induced current can be transferred from the coil 146 to the shaft body 140, and thus to the second electrode 134, via the core distal end 156b and the second cap 164. Similarly, the induced current can be transferred directly from the electrical coil 146 to the shaft body 140, and thus to the second electrode 134. The induced current can also be transferred from the electrical coil 146, for instance the coil proximal end 146a of the electrical coil 146, with a wire through the bore 153, and thus to the first electrode 132. The electrical stimulation screw can define the electrical insulator 152 that can be disposed between the first electrode 132 and the second electrode 134, such that electrical current induced by the magnetic field is not transferred directly from the first electrode 132 to the second electrode 134. The electrical coil 146 of the first electrical stimulation anchor can be electrically connected to the second electrode 134 of the first electrical stimulation screw 111a, and the electrical coil 146 of the second electrical stimulation screw 111b can be electrically connected to the second electrode 134 of the second electrical stimulation screw 111b.

Thus, as described above, a method for treating a fracture in a bone can include positioning a bone plate over the bone, such that the fracture is disposed between a first bone fixation hole and a second bone fixation hole along a longitudinal direction. The method can further include inserting a first electrical stimulation anchor into the first bone fixation hole, and inserting a second electrical stimulation anchor into the second bone fixation hole. Further still, the method can include causing an electrical field to be generated between the first and second electrical stimulation anchors. In some cases, causing the electrical field to be generated includes exposing the bone plate to a magnetic field, so as to induce an electrical current in the first electrical stimulation anchor and the second electrical stimulation anchor. The first electrical stimulation anchor can include a first coil wrapped in a first direction, and the second electrical stimulation anchor includes a second coil wound in a second direction opposite the first direction. In some examples, the method for treating the fracture includes connecting a proximal end of the first electrical stimulation anchor to an electrical conductor of the bone plate, and connecting a proximal end of the second electrical stimulation anchor to the electrical conductor of the bone plate, so as to electrically couple the proximal end of the first electrical stimulation anchor with the proximal end of the second electrical stimulation anchor. In such a configuration, the proximal ends of the bone anchors are both in contact with the bone plate, and thus they are on the same electric potential. Furthermore, because the coils can be wound in opposite directions the potentials of the screw shafts (distal ends of the bone anchors) are reverse with respect to the potential of the proximal ends. Thus, if voltages of identical amounts are induced in both bone anchors the difference between the potential of the screw shafts (distal ends of the bone anchors) can be twice the individual voltages induced in the respective bone anchors.

Continuing with the example, the external magnetic field can induce a voltage in the first coil 146, and thus can generate an electric field between the first electrode 132 and the second electrode 134. The head 112 of the first electrical stimulation screw 111a can be electrically connected to the plate 120. The magnetic field can also induce a voltage in the second coil 146 and generate an electric field between the first electrode 132 and the second electrode 134 of the second electrical stimulation screw 111b, which can include the electrical coil 146 that is wrapped in an opposite direction as the electrical coil 146 of the first electrical stimulation screw 111a. Thus, the current flow of the first electrical stimulation screw 111a can be in the opposite direction of the current flow of the second electrical stimulation screw 111b. The head 112 of the second electrical stimulation screw 111b can also be electrically connected with the plate, such that the electrical voltage of the first electrical stimulation screw 111a can be added to the second electrical stimulation screw 111b.

While the techniques described herein can be implemented and have been described in connection with the various embodiments of the various figures, it is to be understood that other similar embodiments can be used or modifications and additions can be made to the described embodiments without deviating therefrom. For example, it should be appreciated that the steps disclosed above can be performed in the order set forth above, or in any other order as desired. Further, one skilled in the art will recognize that the techniques described in the present application may apply to any environment, whether wired or wireless, and may be applied to any number of such devices connected via a communications network and interacting across the network. Therefore, the techniques described herein should not be limited to any single embodiment, but rather should be construed in breadth and scope in accordance with the appended claims.

What is claimed:

1. An electrical stimulation screw configured to generate an electric field in response to a magnetic field, the electrical stimulation screw comprising:
a head including a head body and a head ring that surrounds the head body, the head defining proximal end of the screw;
a tip opposite the head along a central anchor axis, the tip defining a distal end of the screw; and
a shaft that connects the head to the tip, the shaft defining a shaft body that is monolithic with the head body,
wherein the head body defines a cavity configured to receive a driver so as to rotate the head body and the shaft body about the central anchor axis.

2. The electrical stimulation screw as recited in claim 1, wherein the head ring defines a first electrode that defines a first electrically conductive outer surface of the electrical stimulation screw, and the shaft body defines a second electrode that defines a second electrically conductive outer conductive surface of the electrical stimulation screw that is electrically isolated from the first electrically conductive outer surface.

3. The electrical stimulation screw as recited in claim 2, the electrical stimulation screw further comprising an electrical insulator disposed between the head ring and the head body, so as to electrically isolate the first electrode from the second electrode.

4. The electrical stimulation screw as recited in claim 3, the electrical insulator comprising an epoxy that adheres the head ring to the head body.

5. The electrical stimulation screw as recited in claim 3, wherein the electrical insulator defines the tip.

6. The electrical stimulation screw as recited in claim 3, the electrical stimulation screw further comprising an electrical coil assembly disposed within the shaft body,
wherein the electrical insulator is disposed between the shaft body and the electrical coil assembly.

7. The electrical stimulation screw as recited in claim 6, wherein the electrical coil assembly comprises:
a ferromagnetic core disposed within the electrical insulator; and
an electrical coil wound around the ferromagnetic core so as to contact the electrical insulator.

8. The electrical stimulation screw as recited in claim 7, wherein the head body defines a bore to the central anchor axis, and the electrical stimulation screw further comprises a wire through the bore so as to electrically connect the head ring to a first end of the electrical coil.

9. The electrical stimulation screw as recited in claim 7, wherein the electrical insulator is also disposed within the bore so as to electrically isolate the wire from the head body.

10. The electrical stimulation screw as recited in claim 7, wherein the electrical coil defines a second end opposite the first end, the second end electrically connected to a distal end of the ferromagnetic core that is proximate to the tip.

11. The electrical stimulation screw as recited in claim 10, the electrical stimulation screw further comprising a locking cap disposed between the second end of the electrical coil and the tip along the central anchor axis, the locking cap in contact with the distal end of the ferromagnetic core and the shaft body so as to electrically connect the ferromagnetic core with the shaft body.

12. The electrical stimulation screw as recited in claim 1, wherein the first electrically conductive surface of the head ring includes threads so as to be configured to threadedly mate with a bone implant and secure the bone implant to a bone.

13. The electrical stimulation screw as recited in claim 1, wherein the head ring defines a first head ring surface that faces the central anchor axis, and the head body defines a first head body surface that faces away from the central anchor axis, such that the first head ring surface and the first head body surface face each other and are spaced from each other along a direction radially outward from the central anchor axis.

14. The electrical stimulation screw as recited in claim 13, wherein the first head body surface converges toward the central anchor axis.

15. The electrical stimulation screw as recited in claim 14, wherein the first head body defines a first truncated cone that includes the first head body surface, the truncated cone defining a base diameter at the proximal end, and a frustum diameter proximate to the shaft that is less than the base diameter.

16. The electrical stimulation screw as recited in claim 15, wherein the head ring defines a second truncated cone sized to receive the first truncated cone, such that the first truncated cone is configured to absorb force applied to the head ring in an axial direction from the distal end to the proximal end.

17. The electrical stimulation screw as recited in claim 13, wherein the head body further defines a stop cap that includes a second head body surface that faces the tip, and the head ring defines a second head ring surface that faces the second head body surface, such that the second head body surface and the second head ring surface are spaced from each other along the central anchor axis, and such that the stop cap is configured to absorb force applied to the head ring in an axial direction from the distal end toward the proximal end.

18. The electrical stimulation screw as recited in claim 17, the electrical stimulation screw further defining an insulative epoxy that adheres the first head body surface to the first head ring surface, and the second head body surface to the second head ring surface.

19. A method of fabricating an electrical stimulation screw defining a head, a tip, and a shaft that connects the head to the tip, the method comprising:
winding an electric coil around a ferromagnetic core to define an electrical coil assembly;
inserting the electrical coil assembly into a cavity defined by a shaft body of the shaft;
positioning a head ring around a head body of the head that is monolithic with the shaft body so as to define a gap between the head ring and the head body; and
injecting a non-conductive polymer into the gap between the head ring and the head body, so as to adhere the head ring to the head body.

20. The method of fabricating the electrical stimulation screw as recited in claim 19, the method further comprising:
drilling a bore in the head body; and
placing a wire within the bore so as to electrically connect the head ring with the electrical coil assembly.
